SEQUENCE LISTING (14 sequences)

SEQ ID NO: 1 - DNA, 94 bp, Artificial Sequence
  Description of Artificial Sequence Synthetic oligonucleotide
gcgtatcagctcactcaaaggcggtaatacggttatccacagaatcaggggataacgcaggaaagaacatcgaaccaattctcatgtttgacag

SEQ ID NO: 2 - DNA, 91 bp, Artificial Sequence
  Description of Artificial Sequence Synthetic oligonucleotide
tgataatctcatgaccaaaatcccttaacgtgagttttcgttccactgagcgtcagaccccgtagaaaaggataagcaggacacagcaatc

SEQ ID NO: 3 - DNA, 26 bp, Artificial Sequence
  Description of Artificial Sequence Synthetic oligonucleotide
gtcgagatatgacggtgttcacaaag

SEQ ID NO: 4 - DNA, 25 bp, Artificial Sequence
  Description of Artificial Sequence Synthetic oligonucleotide
tggccatgaatggcgttggatgccg

SEQ ID NO: 5 - DNA, 96 bp, Artificial Sequence
  Description of Artificial Sequence Synthetic oligonucleotide
aaagataaagccacctttgcctagtgttacgaaactgacagaggatagatggaacaagccccagaagaccgtcgagatatgacggtgttcacaaag

SEQ ID NO: 6 - DNA, 95 bp, Artificial Sequence
  Description of Artificial Sequence Synthetic oligonucleotide
cactattctttagttcctgactccaatactgtaggagattccaccaatatttgagggcttcccacccccttggccatgaatggcgttggatgccg

SEQ ID NO: 7 - DNA, 22 bp, Artificial Sequence
  Description of Artificial Sequence Synthetic primer
gataaagccacctttgcctagt

SEQ ID NO: 8 - DNA, 20 bp, Artificial Sequence
  Description of Artificial Sequence Synthetic primer
ttctaggtctcgagatactg

SEQ ID NO: 9 - DNA, 92 bp, Artificial Sequence
  Description of Artificial Sequence Synthetic primer
aaagataaagccacctttgcctagtgttacgaaactgacagaggatagatggaacaagccccagaagaccaagggccacagagggagccata

SEQ ID NO: 10 - DNA, 90 bp, Artificial Sequence
  Description of Artificial Sequence Synthetic primer
cactattctttagttcctgactccaatactgtaggagattccaccaatatttgagggcttcccaccccctgcgtcccagaagttccacaa

SEQ ID NO: 11 - DNA, 22 bp, Artificial Sequence
  Description of Artificial Sequence Synthetic primer
gtgccccggctggttttgcgat

SEQ ID NO: 12 - DNA, 21 bp, Artificial Sequence
  Description of Artificial Sequence Synthetic primer
gtctggcctgtaccgtcagcg

SEQ ID NO: 13 - DNA, 10576 bp, Artificial Sequence
  Description of Artificial Sequence Synthetic polynucleotide
gaaccagatctgatggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaccctgtgagcctgcatggaatggatgacccggagagagaagtgttagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggccctgttgacaattaatcatcggcatagtatatcggcatagtataatacgacaaggtgaggaactaaacccaggaggcagatcatgagtctgaaagaaaaaacacaatctctgtttgccaacgcatttggctaccctgccactcacaccattcaggcgcctggccgcgtgaatttgattggtgaacacaccgactacaacgacggtttcgttctgccctgcgcgattgattatcaaaccgtgatcagttgtgcaccacgcgatgaccgtaaagttcgcgtgatggcagccgattatgaaaatcagctcgacgagttttccctcgatgcgcccattgtcgcacatgaaaactatcaatgggctaactacgttcgtggcgtggtgaaacatctgcaactgcgtaacaacagcttcggcggcgtggacatggtgatcagcggcaatgtgccgcagggtgccgggttaagttcttccgcttcactggaagtcgcggtcggaaccgtattgcagcagctttatcatctgccgctggacggcgcacaaatcgcgcttaacggtcaggaagcagaaaaccagtttgtaggctgtaactgcgggatcatggatcagctaatttccgcgctcggcaagaaagatcatgccttgctgatcgattgccgctcactggggaccaaagcagtttccatgcccaaaggtgtggctgtcgtcatcatcaacagtaacttcaaacgtaccctggttggcagcgaatacaacacccgtcgtgaacagtgcgaaaccggtgcgcgtttcttccagcagccagccctgcgtgatgtcaccattgaagagttcaacgctgttgcgcatgaactggacccgatcgtggcaaaacgcgtgcgtcatatactgactgaaaacgcccgcaccgttgaagctgccagcgcgctggagcaaggcgacctgaaacgtatgggcgagttgatggcggagtctcatgcctctatgcgcgatgatttcgaaatcaccgtgccgcaaattgacactctggtagaaatcgtcaaagctgtgattggcgacaaaggtggcgtacgcatgaccggcggcggatttggcggctgtatcgtcgcgctgatcccggaagagctggtgcctgccgtacagcaagctgtcgctgaacaatatgaagcaaaaacaggtattaaagagactttttacgtttgtaaaccatcacaaggagcaggacagtgctgacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcatctagagtcgacctgcaggcatgcaagcttcagctgctcgaggccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactat

SEQ ID NO: 14 - DNA, 16213 bp, Artificial Sequence
  Description of Artificial Sequence Synthetic polynucleotide
gaaccagatctgatggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaccctgtgagcctgcatggaatggatgacccggagagagaagtgttagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggccctgttgacaattaatcatcggcatagtatatcggcatagtataatacgacaaggtgaggaactaaacccaggaggcagatcatgagtctgaaagaaaaaacacaatctctgtttgccaacgcatttggctaccctgccactcacaccattcaggcgcctggccgcgtgaatttgattggtgaacacaccgactacaacgacggtttcgttctgccctgcgcgattgattatcaaaccgtgatcagttgtgcaccacgcgatgaccgtaaagttcgcgtgatggcagccgattatgaaaatcagctcgacgagttttccctcgatgcgcccattgtcgcacatgaaaactatcaatgggctaactacgttcgtggcgtggtgaaacatctgcaactgcgtaacaacagcttcggcggcgtggacatggtgatcagcggcaatgtgccgcagggtgccgggttaagttcttccgcttcactggaagtcgcggtcggaaccgtattgcagcagctttatcatctgccgctggacggcgcacaaatcgcgcttaacggtcaggaagcagaaaaccagtttgtaggctgtaactgcgggatcatggatcagctaatttccgcgctcggcaagaaagatcatgccttgctgatcgattgccgctcactggggaccaaagcagtttccatgcccaaaggtgtggctgtcgtcatcatcaacagtaacttcaaacgtaccctggttggcagcgaatacaacacccgtcgtgaacagtgcgaaaccggtgcgcgtttcttccagcagccagccctgcgtgatgtcaccattgaagagttcaacgctgttgcgcatgaactggacccgatcgtggcaaaacgcgtgcgtcatatactgactgaaaacgcccgcaccgttgaagctgccagcgcgctggagcaaggcgacctgaaacgtatgggcgagttgatggcggagtctcatgcctctatgcgcgatgatttcgaaatcaccgtgccgcaaattgacactctggtagaaatcgtcaaagctgtgattggcgacaaaggtggcgtacgcatgaccggcggcggatttggcggctgtatcgtcgcgctgatcccggaagagctggtgcctgccgtacagcaagctgtcgctgaacaatatgaagcaaaaacaggtattaaagagactttttacgtttgtaaaccatcacaaggagcaggacagtgctgacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcatctagagtcgacctgcaggcatgcaagcttcagctgctcgaggccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatcatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

(19) United States
(12) Patent Application Publication
(10) Pub. No.: US 2011/0027313 A1
(43) Pub. Date: Feb. 3, 2011

(54) VIRAL RECOMBINEERING AND USES THEREOF

(75) Inventors: Rebeca Geffin, North Miami Beach, FL (US); Richard Myers, Miami Beach, FL (US)

Correspondence Address: VENABLE LLP, P.O. BOX 34385, WASHINGTON, DC, 20043-9998, US

(73) Assignee: University Of Miami, Miami Beach, FL (US)

(21) Appl. No.: 12/936,165

(22) PCT Filed: Apr. 6, 2009
(86) PCT No.: PCT/US09/39660

§ 371 Date: Oct. 1, 2010

(51) Int. Cl. A61K 39/12; C12N 15/74; C12N 7/01; C40B 40/02; A61P 31/12

(52) U.S. Cl. 424/205.1; 435/477; 435/235.1; 506/14

(57) ABSTRACT

The invention uses recombinant technology to create infectious molecular clones that capture the sequence diversity of viral genes found in natural populations of mixed genotype viruses, such as arises during HIV infections and many other viral diseases. The invention captures the sequence diversity of different genes in these “quasi-species” populations by recombining them into in a constant genetic “backbone” for each viral species by backcrossing PCR products derived from quasispecies gene variants into this backbone in an E. coli BAC plasmid.

### BACKGROUND

The 9,000 base pair HIV genome is a relatively compact collection of essential yet highly plastic genes. Due to intrinsic features of the HIV life cycle, an astonishing amount of genetic diversity can be generated in an infected individual in a relatively short period of time. Thus, from an infection that initially emerges from one dominant genotype, a collection of viral “quasispecies” are created and this diverse population is responsible for evasion of the immune response, resistance to anti-retroviral therapy and disease progression (1). To assess the contributions of individual viral genotypes within a collection of viral variants to disease progression and evolved resistance to the immune system and drugs, it is necessary to set up a system in which these variants can be accurately and efficiently sampled and evaluated. It is particularly important when studying the impact of specific sequence variants on viral fitness that assays be performed with an accurate representation of the viral quasispecies diversity present in an infected individual at a particular time.

It has been previously shown that development of resistance to antiretrovirai drugs and to the host immune response can result in reduced viral fitness (4, 5), so quantitative knowledge about the influence of resistance mutations on fitness may reveal new strategies to combat AIDS. Viral fitness has been modeled in the laboratory by the ability of a virus to infect new cells and replicate. Fitness of viral variants can be monitored by competitive growth kinetics in mixed infections with control strains (5). However, since many if not all viral proteins could contribute to viral fitness, it is necessary to dissect the role of individual protein variants in the context of an otherwise constant HIV genetic background. This can be accomplished by backcrossang sequences isolated from viruses in infected patients into a defined genome sequence.

In order to accomplish such a task, it is necessary to have a replicating viral vector into which the gene encoding for a protein of interest can be inserted. We describe the use of a novel approach for manipulation of the HIV genome by bacteriophage-mediated recombineering that can be used to address the problems associated with researching and developing treatments for viruses with high sequence diversity such as HIV.

### SUMMARY

The invention uses recombinant technology to create infectious molecular clones that capture the sequence diversity of viral genes found in natural populations of mixed genotype viruses, such as the diversity that arises during HIV infections and many other viral diseases. The invention captures the sequence diversity of different genes in these “quasi-species” populations by recombining them into in a constant genetic “backbone” for each viral species by backcrossing PCR products derived from quasispecies gene variants into this backbone in an E. coli BAC plasmid. In the examples set forth herein, we describe the use of an HIV BAC bearing Δ(env)::galK to capture env gene variants that were PCR amplified from patient samples. While recombineering of env variants is described as an example, any viral gene or genetic sequence variants can be exchanged in this manner, thereby greatly extending the utility of the method. Another counter-selectable marker with superior performance has also been provided herein. This new selection uses a three component “RTS cassette” which includes the rpsl, tetA and sacB genes.

Libraries of gene variants backcrossed into constant genetic backbones provide the means to assess viral fitness changes during the course of an infection and under selective pressure from the host immune system or by drug therapies. These libraries can also be used to evaluate the effectiveness of newly developed antiviral drugs since they will better reflect the genetic diversity of viral populations in infected people.

This inventive process, exemplified using the HIV genome, can be extended to other viruses, especially those with high genomic diversity, such as, but not limited to: Hepatitis A, Hepatitis B, Hepatitis C, polio and influenza viruses; where the representation of the quasispecies found in vivo is important for the following purposes: the study of viral proteins their characteristics, function, and their contribution to viral fitness, for the sensitivity of viral proteins to new drugs, for the design of vaccines or therapeutic approaches, for the design of tools to evaluate vaccine efficacy, and for the better understanding of the virus pathogenesis.

Embodiments of the invention are directed to a method of generating viral recombinants with selected viral gene(s) using the following step: introducing said selected viral gene(s) into E. coli containing a plasmid or a defective λ prophage expressing exo, bet and gam, optionally in the presence of a bacteriophage λ heat sensitive cI repressor and a BAC plasmid containing a reference genome for the viral target of recombineering; and exposing said E. coli to conditions such that recombination occurs between the introduced viral gene(s) and the target genome. Such methods can also use a positive or negative selection marker. For example, the plasmid can include a RTS cassette or galK gene. Recombinant viruses made using these methods can be used in libraries, as vaccines, or in assays to test vaccine efficacy and/or viral fitness.

### DESCRIPTION OF THE FIGURES

FIG. 1 shows an example of single stranded oligo recombineering that was performed as described herein.

FIG. 2 shows a gel illustrating an example of pJJ5 & 8 clones pre-digestion. The lanes shown are: 1) pJJ5 2-5) pJJ5/BAC multimers from 4 hr recovery 6-9) pJJ5/BAC multimers from 20 hr recover.

FIG. 3 shows a gel illustrating an example of using a digest step to resolve multimers with AatII. The lanes shown are: 1-4) digest of lanes 2)6)7)8) in FIG. 2 with AatII.

FIG. 4 shows a gel illustrating an example of a restriction digest with NcoI. The lanes shown are: 1) pJJ5 2) pJJ5 digest w/AatII 3) pJJ5 digest w/NcoI 4) pLL11 5) pLL11 digest with AatII 6) pLL11 digest w/NcoI. The recombination frequency was about 1×10^{-4.}

FIG. 5 shows a gel illustrating an example of a restriction digest with pLL3. The lanes shown are: 1) pLL3 2) pLL3 digest w/NcoI 3) pLL3 digest w/AatII 4) pLL3 digest with HpaI 5) pLL3 digest w/Mlul and BmtI.

FIG. 6 shows a gel illustrating an example of PCR amplification of RTS cassette. The lanes shown are: 1) negative control 2) negative control 3) pRTS amplified with rts-F and rts-R 4) pLL11 amplified with rts-F and rts-R 5) pLL3 amplified with rts-F and rts-R. The recombination example used coelectroporation of between 2-6×105 and was plated on 15 μg/ml let to generate a recombinant frequency of 2-6×10⁵ plated on 15 μg/ml tet.

FIG. 7 shows a gel illustrating an example of high and low molecular weight species. The lanes shown are: 1) low MW clone 2) pLL2 3) Low MW clone 4) pLL2 5) low MW clones 6) Low MW clone 7) pLL2.

FIG. 8 shows a gel illustrating an example of a restriction digest high and low MW species. The lanes shown are: 1) pLL3 2) pLL3 digested w/NcoI 3) Low MW “pLL2” 4) Low MW “pLL2” digested with NcoI 5) pLL2 clone-2 6) pLL2 clone-2 digested w/NcoI 7) pLL 2 clone-7 8) pLL2 clone-7 digested w/NcoI.

FIG. 9 shows the sequence inferred for plasmid pLG2 (SEQ ID NO: 13).

FIG. 10 shows the sequence inferred for plasmid pLL1 (SEQ ID NO: 14).

### DETAILED DESCRIPTION

We describe the use of a novel approach for manipulation of the HIV genome by bacteriophage-mediated recombineering. This process, exemplified using the HIV genome, can be extended to other viruses, can be used, for example, to study viral proteins including their characteristics, function, and contribution to viral fitness. This process can also be used to test for the sensitivity of viral proteins to new drugs, for the design of vaccines or therapeutic approaches, for the design of tools to evaluate vaccine efficacy, and for the better understanding of the virus pathogenesis.

In recombineering, homologous recombination between short target sequences is promoted in bacteria using phage recombinases native to that bacterium. In the current invention, this is accomplished, for example, through the utilization of the “Red” recombination enzymes encoded in the bacteriophage lambda genome: an exonuclease (λexo, the product of the reda), a single-stranded DNA (ssDNA) binding protein (β protein, the product of bet), and an inhibitor of the host RecBCD exonuclease. The Red genes are integrated into a host bacterial genome under control of a thermolabile repressor (cI⁸⁵⁷) and can be easily induced and expressed when homologous recombination is desired. Once induced, recombination between a cloned cDNA copy of an HIV-1 prototype virus and a PCR product flanked by targeting regions of homology can be accomplished in this bacterium. Such a recombination event is mediated through resection of double-stranded DNA ends by λexo and coordinated loading of β protein onto the nascent ssDNA to form a nucleoprotein complex which promotes homologous pairing and strand exchange with homologous target sequences.

Using this recombineering system, we first created a prototypical HIV-1 bacterial artificial chromosome, which includes the entire HIV-1 genome, a BAC origin of replication for propagation and manipulation of a single copy HIV clone in bacteria, and a selectable drug resistance marker. Next, we used recombineering to introduce a counter selectable marker linked 100% to a deletion of the HIV-1 env gene in the prototypical HIV-1 BAC.

Two different counter-selectable markers have been employed. The first, was based on galK, was described in U.S. Provisional Patent Application 61/064,964, which is herein incorporated by reference in its entirety. We have subsequently evaluated another counter-selectable marker with improved performance. This new selection uses a three component “RTS cassette” which includes the rpsl, tetA and sacB genes. Positive selection for introducing the RTS cassette is conferred through tetracycline resistance encoded by the tetA gene, whose protein product is a membrane pump that pumps tetracycline out of the cell (6). Negative selection for crossing out the RTS cassette while crossing in the env gene by recombineering is conferred by all three components.

First, streptomycin sensitivity is achieved through production of the RpsL protein, which associates with the 30S subunit of the ribosome and exerts a dominant streptomycin sensitive phenotype in a normally streptomycin resistant background (6). Second, the sacB gene product (levansucrase) confers sensitivity to sucrose in the medium by converting sucrose to levan which accumulates in the periplasmic space and causes cell death (6). Finally, both TetA and RpsL are overexpressed by being fused in a synthetic operon to the ompF promoter. In LB medium lacking NaCl, high level transcription is induced, resulting in TetA-dependent weakening of the cytoplasmic membrane leading to sensitivity to hypo-osmotic pressure and increasing cell permeability to streptomycin and kanamycin, both of which target RpsL. Altogether, the strength of this negative selection cassette is about equal to the strength of the positive selection, a great improvement over the previous selection methods we have used (galk: Galactose utilization and 2-deoxygalactose sensitivity, Lindley, Lu, Schmier, Geffin, Myers, unpublished; tetA: tetracycline resistance and fusaric acid sensitivity, Chen, Myers, unpublished; Gat-Sac: chloramphenicol resistance and sucrose sensitivity, Recalde, Hakimpour, Myers, unpublished). After introducing the “RTS” selection cassette, a PCR amplified sequence for the missing gene (e.g. env) can be amplified from a laboratory control virus or patient sample, and this can then be used to reconstitute a complete HIV genome, suitable for functional studies.

The invention described herein includes novel methods of generating viral recombinants with selected viral gene(s), capturing the quasispecies diversity of such gene sequences. These methods can be performed by introducing a selected viral gene or viral genes into E. coli containing a plasmid or a defective λ prophage expressing exo, bet and gam. In some embodiments, this step can be performed in the presence of a bacteriophage λ heat sensitive cI repressor and a BAC plasmid containing a reference genome for the viral target of recombineering. After the viral genome has been introduced, the E. coli can be treated under conditions such that recombination occurs between the introduced viral gene(s) and the target genome.

The plasmid in these methods can vary as experimental conditions dictate. In some embodiments, the plasmid is an HIV plasmid deleted in any viral gene or any viral sequence and the selected viral gene(s) is the same gene or same viral sequence. The HIV plasmid can vary, pJJ5 is used as an example, but other HIV molecular clones could be used as well.

The target plasmid can have a RTS cassette inserted at the site of a deletion that removed the gene that is to be replaced during recombination. In some embodiments, an HIV gene replaces the RTS cassette during recombination. In some embodiments, the RTS cassette contains a positively selectable trait conferred through tetracycline resistance encoded by the tetA gene. As one of skill in the art will appreciate, in some embodiments, other positive and negatively selectable markers (e.g., genes conveying temperature or drug resistance) can be used as part of the cassette and still retain the spirit of the invention described herein.

The target plasmid can also have a galK⁺ gene inserted at the site of a deletion that removed the gene that is to be replaced during recombination. In some embodiments, an HIV gene replaces the galK⁺ gene during recombination.

in some embodiments, the plasmid can be any virus having a high genomic diversity, For example, the plasmid can be, but is not limited to, any of the following viruses: HIV, Hepatitis A, Hepatitis B, Hepatitis C, polio or influenza. As one of skill in the art will appreciate, this list is provided by way of example and is not intended to be limiting.

The invention described herein is also directed to recombinant viruses obtained using the methods described herein. These recombinant viruses can be used to screen for therapeutic agents. For example, a culture with one or more of the recombinant viruses can be subjected to a therapeutic agent or combination of therapeutic agents. The resulting viral culture growth can be compared to a control sample to assess the effectiveness of the therapeutic agent. In some embodiments, such screens can be run using high-throughput techniques or using more conventional cellular assays.

The recombinant viruses generated herein can also be used to generate therapeutic agents such as vaccines. For example, a recombinant virus produced herein can be inactivated and introduced into a test animal to measure its ability to trigger an effective immune response. Such assays are well know to one of skill in the art and have not been specifically included herein. Those recombinant viruses found to trigger an immune response can then be formulated into vaccines.

The recombinant viruses can also be used to test vaccines, for example an HIV vaccine, in a manner similar to those described for therapeutic agents in general. However, such assays can be modified to allow the researchers to more precisely measure the immune response than would be necessary for assays examining small molecule therapeutics that have been designed, for example, to be toxic to the virus. Accordingly, these assays and test methods can be used to measure vaccine efficacy.

For example, the following assay can be used to develop vaccines and to test for viral efficacy. Proponents of HIV vaccines advocate for the use of immunogens that elicit both cytotoxic T cell responses, as well as neutralizing antibody responses. Due to the high variability of the HIV genome, especially in the envelope gene, which is the target for neutralizing antibodies, vaccinees' sera may neutralize some viruses but not others. Currently, the ability of vaccinees' sera to neutralize HIV infectivity is being assessed with a panel of viruses, but those may not be representative of the viruses that the vaccinees are exposed to in a natural infection. Recombinant viruses prepared in the manner described in this patent can be generated that contain the envelope quasispecies diversity predominant in a given geographical region. Such viruses can be used for assays designed to test the neutralization properties of vaccinees' sera to better estimate their ability to prevent infection with viruses these individuals might be exposed to. Accordingly, theses vaccine development and testing methods are part of some embodiments of the invention.

Some embodiments of the invention are directed to the following assays for testing viral fitness. Adaptations that allow the virus to persist in the host may be crucial for the virus' survival, but they may come in exchange for reduced fitness. Examples of this compromise are found in the outcomes of drug treatments with reverse transcriptase and protease inhibitors. A decline in viral fitness after drug treatment was first described in 1996 with one patient treated with lamivudine (3TC), revealing that the kinetics of viral replication was lower in virus variants that were resistant to 3TC than in non-resistant viruses (Back et al, 1996). Since that study was published, multiple other studies have addressed the same issues and have found similar results, indicating that resistance to a variety of antiretrovirals such as protease inhibitors (Croteau et al, 1997; Martinez-Picado et al, 1999) reverse transcriptase (Harrigan, Bloor, and Larder, 1998; Maeda, Venzon, and Mitsuya, 1998) and also entry inhibitors that target the viral glycoprotein (Lu et al, 2004) (Armand-Ugon et al., 2003) are accompanied by a reduced fitness of the surviving virus. It is important to note that selection of variants with increased fitness provides evolutionary advantage and often is the difference that allows the virus to survive in the circumstances present in the host. For example, a virus that is resistant to antiretroviral drugs even if it has reduced replicative capacity is more fit in the presence of that drug than a sensitive virus with high replicative capacity. In the absence of drug, the virus with higher fitness is the one with higher replication rates.

Viral replicative capacity of viruses can be estimated single infections of PBMC (Campbell et al, 2003). Viral replication capacity can be also measured using the test virus in competition with another virus isolate which is different enough from the test viruses, for example, a virus from a different clade. Virus production of each virus in competition experiments can be monitored by TaqMan real time PCR using oligonucleotide primers that can discriminate between the two competing viruses (Weber et al., 2003). The latter method is more sensitive, and small fitness differences can be detected, while the single infection assay detects larger differences between isolates. Recombinant viruses prepared using the methods described in this patent could be useful tools to test for viral fitness as a result of resistance to any antiviral intervention (drugs or immunotherapy), including existing and new drugs that target any of the viral proteins.

The following examples are further illustrative of the present invention, but are not to be construed to limit the scope of the present invention.

### EXAMPLES

The following materials and methods were used in the examples shown herein.

The following strains were used herein:_RIK386 (AKA SW102ΔtetA): a derivative of DH10B. mcrA Δ(mrr-hsdRMS-mcrBC) ΔlacX74 deoR endA1 araD139 Δ(ara, leu) 7697 rpsL recA1 nupGφ80dlacZΔM15 [λc1857 (cro-bioA)<>Tet] ΔgalKΔtetA. (The tetA deletion accomplished using single stranded oligo recombineering described below.)

One Shot® TOP10 (Invitrogen): F- mcrA Δ(mrr-hsdRMS-mcrBC) φ80lacZΔM15 ΔlacX74 recA1 araD139 Δ(araleu)7697 galU galK rpsL (StrR) endA1 nupG

The plasmids listed in the table below were used herein:

| | | | | Stock |
|---|---|---|---|---|
| Plasmid | Source | Genotype | Method | Strain |
| pJJ5 | Jose A. Este, | HIV-1:HXB2Δenv (Amp^{R}) SP73 ori | Cloning | Top10 |
| | Fundaucio Irsicaixa, | | | |
| | Barcelona Spain | | | |
| pBeloBAC11 | NEB | (Cml^{R}) BAC ori | Gift | Top10 |
| pLL11 | This study | pJJ5ΔPSP73ori[BACori] | Recombineering | Top10 |
| | | (Cml^{R}, Amp^{R}) | | |
| pRTS | Craig Strathdee | BAC ori (Cml^{R}, Tet^{R}, Suc^{S}, Strep^{S}) | Gift | Top10 |
| pLL3 | This study | pJJ5/BACΔenv::RTS BAC ori | Recombineering | Top10 |
| | | (Amp^{R}, Cml^{R}, Tet^{R}, Suc^{S}, Strep^{S}) | | |
| pNL43 | NIH AIDS Research | HIV-1 pUC18 (Amp^{R}) | Gift | Top10 |
| | and Reference Reagent | | | |
| | Program | | | |
| pLL2 | This study | pLL3 RTS::NL433env BACori | Recombineering | Top10 |
| | | (Amp^{R}, Cml^{R}) | | |

The oligonucleotides listed below and in the accompanying sequence listing were used herein.

BAC -pSP73:
F:
(SEQ ID NO: 1)
5′GCGTATCAGCTCACTCAAAGGCGGTAATACGGTTATCCACAGA
ATCA GGG GAT AAC GCA GGA AAG AAC ATCGAACCAATT
CTCATGTTTGACAG3′
R:
(SEQ ID NO: 2)
5′TGATAATCTCATGACCAAAATCCCTTAACGTGAGTTTTCGTTCC
ACTGAGCGTCAGACCCCGTAGAAAAGGATAAGCAGGACACAGCAATC3′
RTS:
F:
(SEQ ID NO: 3)
5′GTCGAGATATGACGGTGTTCACAAAG 3′
R:
(SEQ ID NO: 4)
5′TGGCCATGAATGGCGTTGGATGCCG 3′
Env-RTS:
F:
(SEQ ID NO: 5)
5′AAAGATAAAGCCACCTTTGCCTAGTGTTACGAAACTGACAGAG
GATAGATGGAACAAGCCCCAGAAGACCGTCGAGATATGACGGTGTTCA
CAAAG 3′
R:
(SEQ ID NO: 6)
5′CACTATTCTTTAGTTCCTGACTCCAATACTGTAGGAGATTCCAC
CAATATT TGAGGGCTTCCCACCCCTTGGCCATGAATGGCGTTGGATG
CCG 3′
Env outside primers:
F:
(SEQ ID NO: 7)
5′GATAAAGCCACCTTTGCCTAGT 3′
R:
(SEQ ID NO: 8)
5′TTCTAGGTCTCGAGATACTG 3′
Env nested primers:
F:
(SEQ ID NO: 9)
5′AAAGATAAAGCCACCTTTGCCTAGTGTTACGAAACTGACAGAGGAT
AGATGGAACAAGCCCCAGAAGACCAAGGGCCACAGAGGGAGCCATA3′
R:
(SEQ ID NO: 10)
5′CACTATTCTTTAGTTCCTGACTCCAATACTGTAGGAGATTCCACCA
ATATTTGAGGGCTTCCCACCCCCTGCGTCCCAGAAGTTCCACAA3′
Env internal primers 6877-7892:
F:
(SEQ ID NO: 11)
5′GTGCCCCGGCTGGTTTTGCGAT3′
R:
(SEQ ID NO: 12)
5′GTCTGGCCTGTACCGTCAGCG3′

The following example media were used for selection as described herein.

Selective Media:

LB/Cml: 1% tryptone, 0.5% NaCl, 0.5% yeast extract, 1.5% agar, 12.5 μg/ml chloramphenicol. p LB/Fusaric acid: 0.5% tryptone, 1% NaCl, 0.5% yeast extract, 1% NaH₂PO₄, 40 mg/800 ml chlorotetracycline, 9.6 mg/800 ml fusaric acid diluted in 500 μL DMF, 4 ml 20 mM ZnCl₂, 1.4% agar.

LB/Tet/Cml: 1% tryptone, 0.5% NaCl, 0.5% yeast extract, 1.5% agar, 15 μg/ml tetracycline, 12.5 μg/ml chloramphenicol.

NSLB/Kan/Strep/Sucrose/Cml: 1% tryptone, 0.5% yeast extract, 1.5% agar, 1 μg/ml kanamycin, 500 μg/ml streptomycin, 12.5 μg/ml chloramphenicol, 5% sucrose.

The following methods and parameters were used for PCR amplification of

recombineering substrates:

pBeloBAC11→ BAC amplification: A pBeloBAC11 PCR product was amplified using BAC-pSP73 primers under standard PCR conditions adjusted for enzyme and product length. Amplification was performed using KOD Hot Start DNA Polymerase, a high fidelity, high processivity polymerase manufactured by TOYOBO and distributed by EMD Chemicals. Large quantities of product were generated, visualized using crystal violet and gel purified,

pRTS-BAC→ RTS amplification: The RTS cassette PCR product was amplified using standard PCR conditions adjusted for enzyme and product length. The first round of amplification served to generate an RTS cassette template for further amplification for addition of targeting homology. This first round product was generated using rts-F arid rts-R primers with Roche High Fidelity PCR Master mix. The product was then crystal violet gel purified to eliminate the pBAC-RTS template plasmid. The pure product was then used as a template for an additional amplification with env-rts-f and env-rts-R primers. Second round amplification was performed using KOD Hot Start DNA Polymerase. The final product was desalted and concentrated using Qiagen Gel extraction kit,

pNL43→ envelope amplification: The env gene was amplified using standard PCR conditions adjusted for enzyme and product length. The first round of amplification was performed using env outside primers that lie outside the env sequence and Roche High Fidelity PCR Master mix to generate a template for further amplification with addition of targeting homology. The product was then crystal violet gel purified to eliminate pNL43 template plasmid. The pure product was then used as a template for additional amplification with env nested primers. Second round amplification was performed using KOD Hot Start DNA Polymerase. The final product was desalted and concentrated using the Qiagen Gel extraction kit.

The following recombineering protocol was used in the examples below.

Recombineering Protocol:

Rik386 was struck out from a frozen stock on an LB plate 2 days before desired day for recombineering. A single colony was picked 24 hrs later and used to inoculate 5 ml of LB, and grown at 30° C. in an air shaker at 200 rpm overnight. The fresh overnight culture was diluted 1:100 into fresh LB medium, and grown to midlog phase (OD⁶⁰⁰ between 0.5-0.6). Half of the culture was transferred to a new flask and half was induced in a 42° C. water bath for 15 minutes. (NB: it is important to use a water bath to insure a rapid transition in the culture temperature to induce the Red system.) Both induced and uninduced cultures were then swirled in an ice/water slurry for 10 minutes before being transferred to pre-chilled Sorvall SS34 tubes. Cultures were then centrifuged at 9,000 rpm for 10 minutes, the supernatant was removed and the pellet was resuspended in an equal starting culture volume of ice-cold sterile water. Cultures were centrifuged again under the same conditions, the supernatant removed and the pellet resuspended in 1/10 the original culture volume of ice-cold sterile water. The cell suspension was transferred to 1.5 ml pre-chilled microfuge tubes and centrifuged a final time at 14,000 rpm for 1 minute. The pellets were resuspended in 1/100 the original culture volume with ice-cold sterile water and kept on ice until electroporation (<30 minutes, typically). (For longer storage, the final suspension is made using 10% glycerol to stabilize the cells, albeit at a moderate loss in recombineering efficiency). 40 μl of prepared cells were added to the desired DNA (100 ng-500 ng in a volume not to exceed 5% of the total volume, 1 μl is perfect) and then transferred to a pre-chilled 1 mm gap electroporation cuvette (Bio-Rad). Cells were electroporated at 1750 V, 25 μF, 200 Ω, and recovered immediately into 1 ml of LB, transferred to an air shaker and grown for 1-4 hours at 30° C. with shaking at 200 rpm. Cultures were diluted as necessary plated on selective media; the remaining culture was typically returned to the air shaker tor overnight outgrowth and replating after another 20 hrs of growth, in case recovery or phenotypic expression required more incubation time.

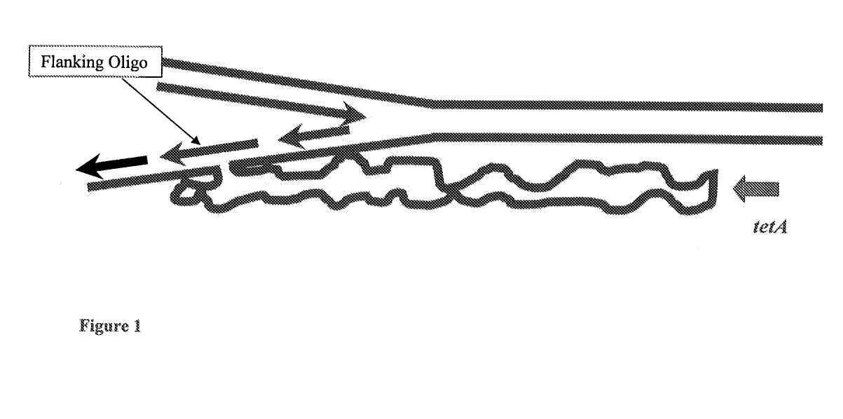

The following methods were used for strain construction for RTS selection. A Tet^{S} strain is needed to carry out RTS^{+/-} selection, so the tetA gene that is linked to the partial λ prophage in SW102 was deleted to generate RIK386. This was accomplished using ssDNA oligo recombineering. This method is different from that of the recombineering used throughout the rest of this patent description, in that the recombination substrate is a ssDNA oligonucleotide as opposed to a dsDNA PCR product. The oligo is composed of the sequences that flank the tetA gene (FIG. 1). In this variation on recombineering, β protein anneals the oligo to the lagging strand template of chromosomal DNA during replication (this protocol does not rely upon λexo, unlike PCR based dsDNA recombineering). Recombinants were counter-selected for Tet^{S} by selecting resistance to fusaric acid and ZnCl₂, and then screened for other markers in SW102. The recombination frequency of this type of recombineering is dependent upon the size of the desired manipulation (typically on the order of 0.1% -50%). In most cases the frequency is so high that it is not necessary to apply a selection to enrich for recombinant clones. For the ˜1 kb tetA deletion, fusaric acid selection gave 80% positive recombinants among the colonies that arose.

### Example 1

### Single Stranded Oligo Recombineering

As shown in FIG. 1, single stranded oligo recombineering was performed. The DNA isolation was done using a PureLink™ Quick Plasmid Miniprep Kit and a PureLink™ Quick Plasmid Midiprep Kit (Invitrogen). The digests of plasmid DNA were performed according to standard protocol, using approximately 3 units enzyme per microgram supercoiled plasmid. Digests carried out overnight at 37° C. All enzymes were from New England Biolabs. Ligation of purified pJJ5/BAC DNA was performed according to standard ligation protocols, at room temperature for 10 minutes with low DNA concentrations used to promote self ligation. Gel purification was performed using a S.N.A.P.™ UV-Free Gel Purification Kit from Invitrogen in all gel purifications. This kit uses crystal violet dye and does not require the use of UV light to visualize bands, thus reducing any harmful effect from exposure to UV light. One Shot® TOP10 competent cells were used in a standard heat shock transformation protocol. Cells were plated and grown at 37° C.

PCR for RTS recombinants was performed using rts-F and rts-R primers with Roche High Fidelity PCR Master mix. PCR for env recombinants was performed using env internal primers 6877 and 7892 with Roche High Fidelity PCR Master mix.

### Example 2

### Double Stranded DNA Recombineering: Exchange of Multi Copy Origin of Replication by a Single Copy Origin of Replication

Plasmid pJJ5 is an env-deleted HIV_{HXB2} plasmid provided by Jose A. Este, Fundaucio Irsicaixa, Barcelona Spain, and was originally prepared by A. de Ronde (de Jong, J. J. Goudsmit, J., Keulen, W., Klaver, B., Krone, W. Tersmette, M., and de Ronde, A. (1992) Human immunodeficiency virus type 1 clones chimeric for the envelope V3 domain differ in syncytium formation and replication capacity. J. Virol. 66(2), 757-765) A 2800 bp portion of the env gene was removed and in its place a 15 bp “stuffer” sequence was inserted. Plasmid pJJ5 was constructed from pSP73 (Promega) which has a multicopy origin of replication, and is maintained at approximately 700 copies/cell. Having a high number of plasmids present at any time poses numerous obstacles to recombineering (7). Most troublesome, multicopy targets yield a mixed population of plasmids after any recombination experiment as recombination efficiency is less than 100% (typically 10⁻⁵-10⁻⁴ with PCR products) and this inhibits selection for recombinants and can make isolating single recombinant plasmids difficult. Also, expression of the Red recombineering system creates concatemeric plasmid multimers (2) making it very difficult to isolate pure monomeric recombinant plasmid clones.

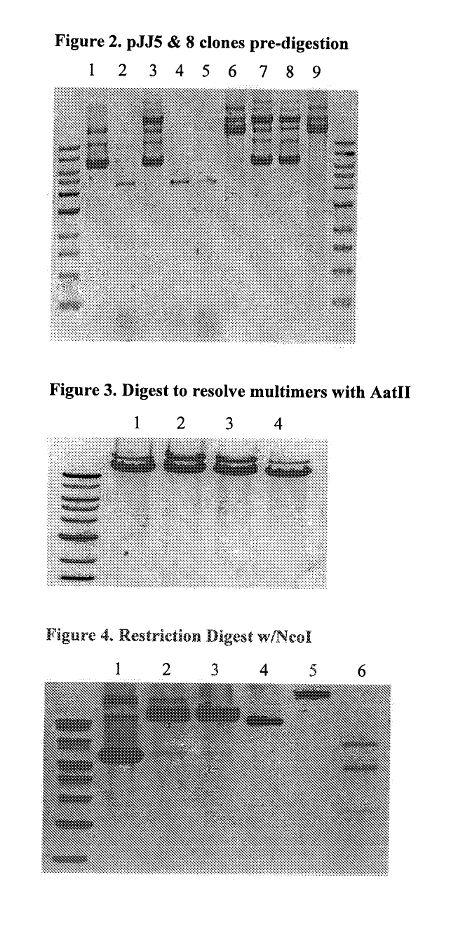

To improve identification of recombinants, a single-copy origin of replication (ori) was isolated by PCR amplification of the ˜6261 bp bacterial artificial chromosome ori and associated genes required for faithful plasmid segregation found in the pBeloBAC11 plasmid. The BAC ori was amplified using primers that add an extra 50 bp of targeting homology to sequences flanking the pSP73 ori to the dsDNA product. Co-electroporation of RIK386 with pJJ5 and the PCR amplified BAC was performed and recombinants were selected on LB+Cml medium (recombination frequency ˜1×10⁻⁴). Eight colonies were struck on the same medium, individual colonies were then again selected and grown in LB+Cml liquid medium overnight, Minipreps to isolate plasmid DNA were then performed and plasmid DNA ran in a 0.8% agarose TAE gel along with starting pJJ5 plasmid DNA (FIG. 2.). Every one of the 8 clones exhibited multiple plasmid populations, all of which had minor or major populations different from the starting material. Due to muitimer formation, a restriction enzyme that cleaved a single site in the plasmid (AatII) was used to liberate pJJ5 from pJJ5/BAC recombinants forming two distinct linear populations (FIG. 3.). The digest was run in a crystal violet agarose gel to allow for visualization without the use of UV light, and the higher molecular weight band excised and purified. The purified product was then ligated at low DNA concentration to promote self-ligation and the product used to transform chemically competent TOP10 cells selecting for Cml^{R}. Colonies were chosen and midipreps prepared in order to isolate large quantities of highly concentrated plasmid DNA for future use. Restriction digests using NcoI were performed to confirm BAC recombinants. The resulting plasmid was called pLL11 (FIG. 4.).

### Example 3

### Introduction of a Selection Marker for Positive/Negative Selection

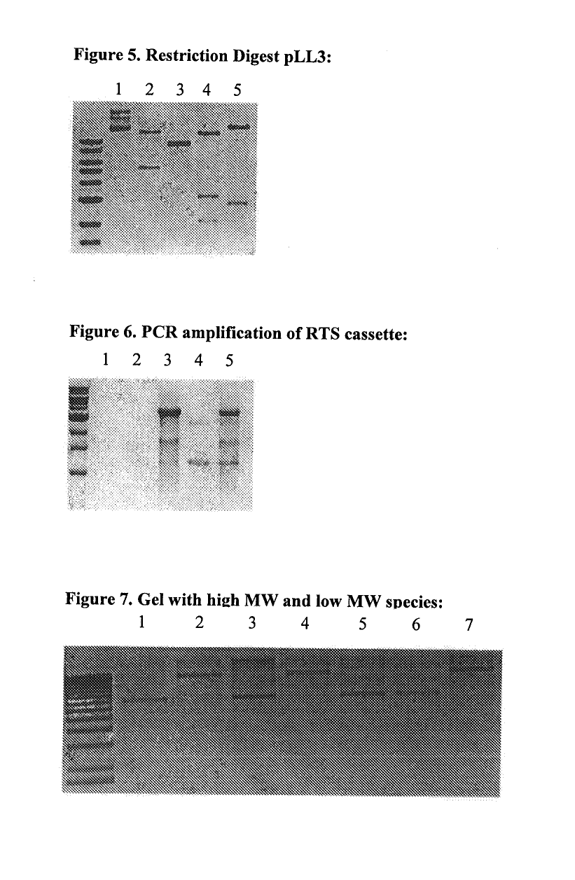

After isolating the HIV-1 BAC (pLL11) recombinant, the 15 bp env stuffer sequence was replaced with the RTS positive/negative selection cassette. The approximately 4 kb RTS was amplified using primers that attach 50 bp of targeting homology to sequences flanking the env deletion in pLL1. The RTS cassette was recombineered into pLL11 by either co-electroporating both the plasmid and the insert at the same time, or by first establishing the plasmid in RIK386 and then adding only the insert. Significant optimization of tetracycline concentration in the selective media was required to find the highest fold difference in plating efficiency for positive recombinants. Titration experiments determined that 18 μg/ml tetracycline gave the greatest fold difference in plating efficiency (about 5 log) between Tet^{S} (non-recombinants) and Tet^{R} recombinants on LB medium made with 5 gm/l NaCl (NB: since tetA is under control of the ompF promoter in the RTS cassette, the salt concentration in the medium is critical), however at this concentration there is a 10-100 fold reduction in plating efficiency for the Tet^{R} recombinants. This reduced plating efficiency results in an apparent reduction in recombination frequency and should be accounted for when calculating recombineering frequencies. Colonies were chosen and restruck on selective media, so that isolated colonies could be chosen for growth in liquid media, DNA isolation and characterization. Plasmid DNA was digested with multiple restriction enzymes and also PCR amplified to identify positive clones (FIGS. 5-6). The resulting plasmid (pLL3) is an HIV-1 BAC with the RTS cassette replacing the env gene. Midipreps of pLL3 were prepared to obtain highly concentrated plasmid for future use.

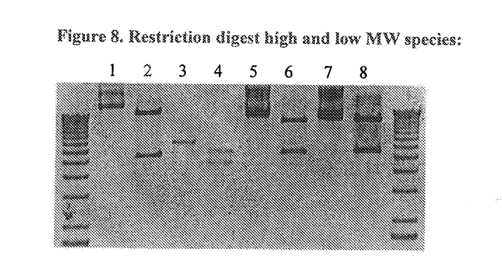

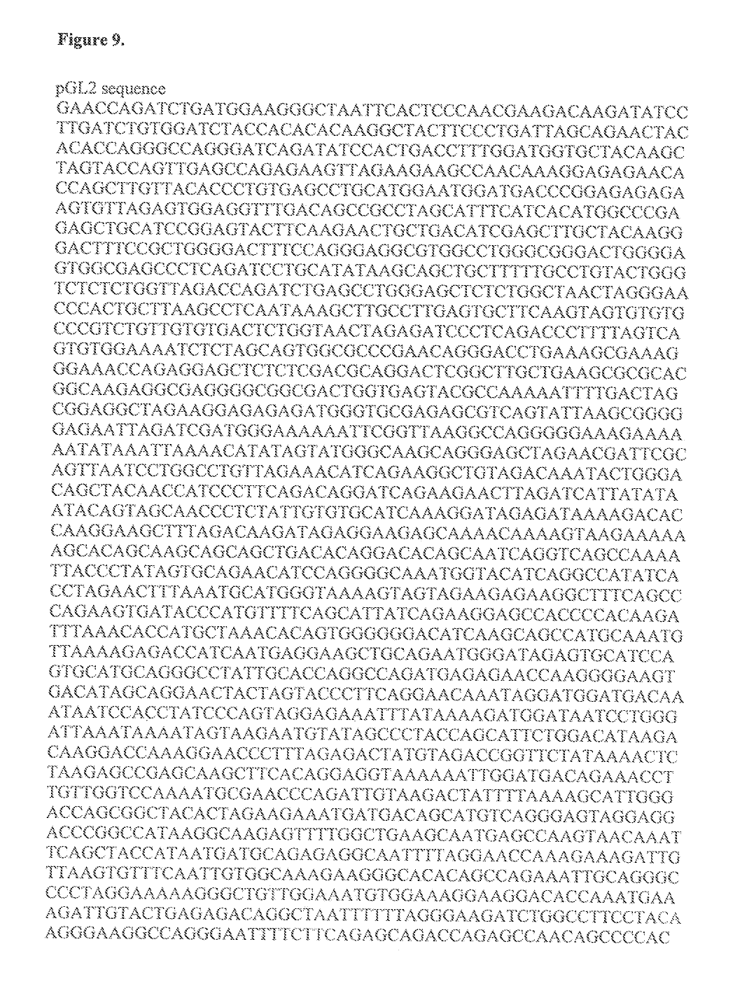

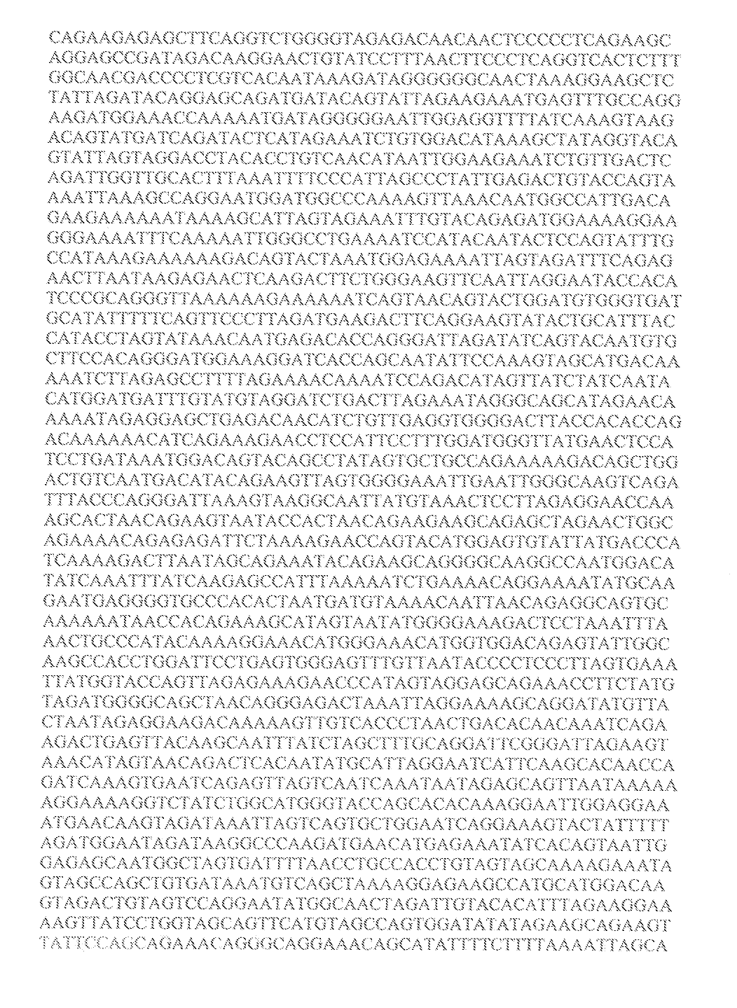

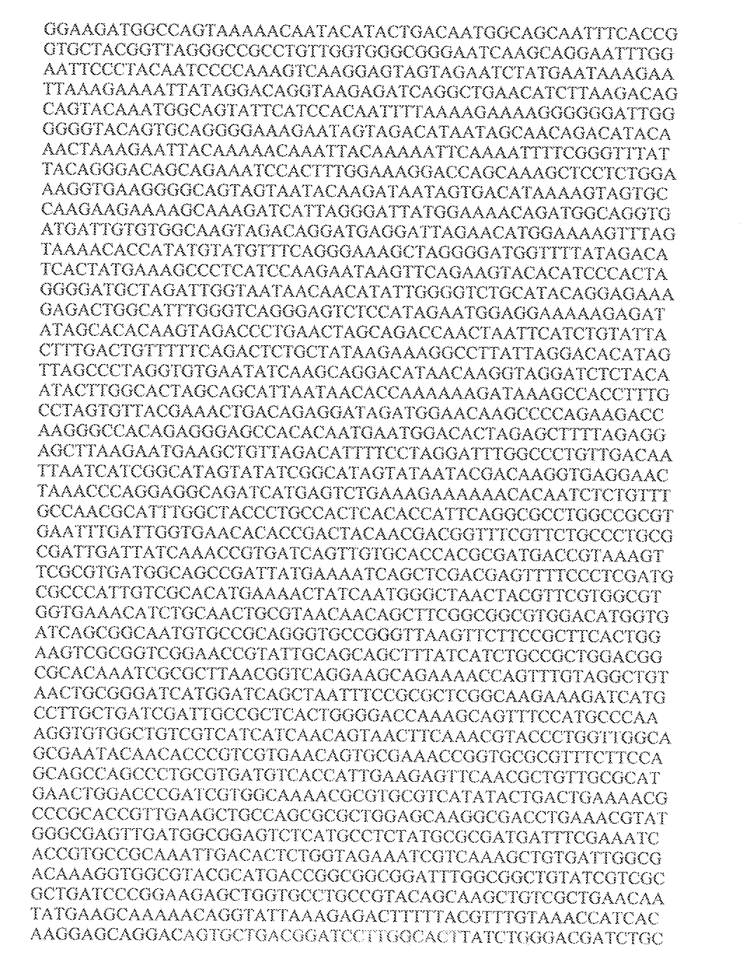

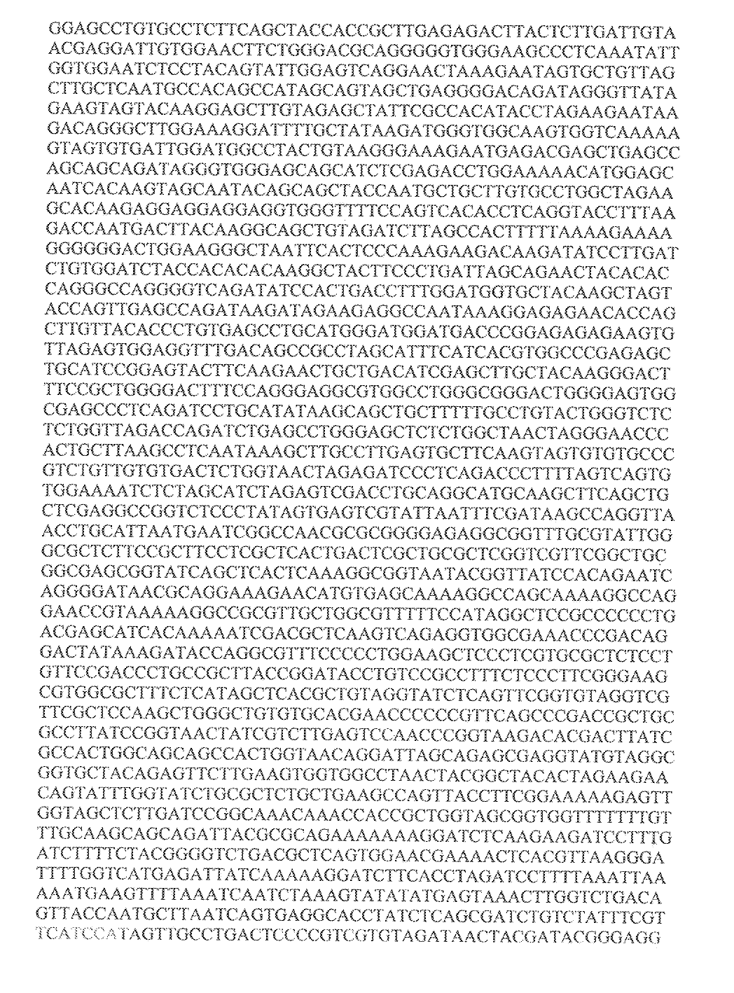

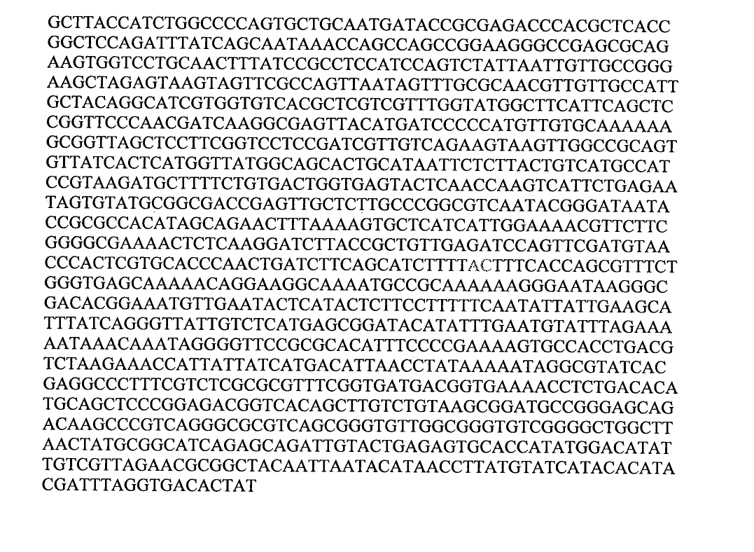

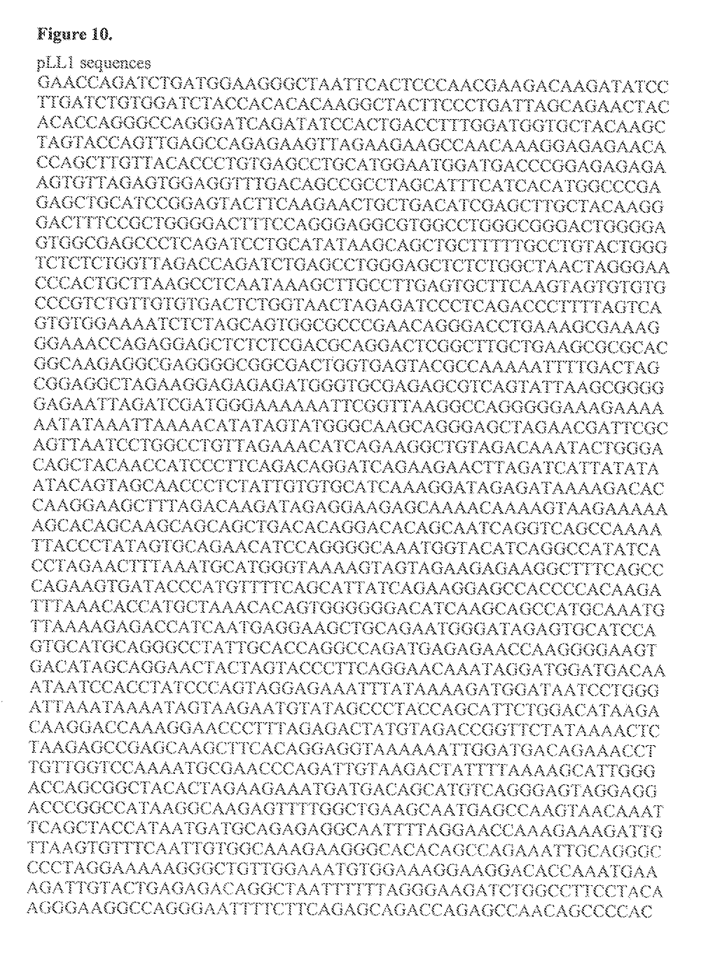

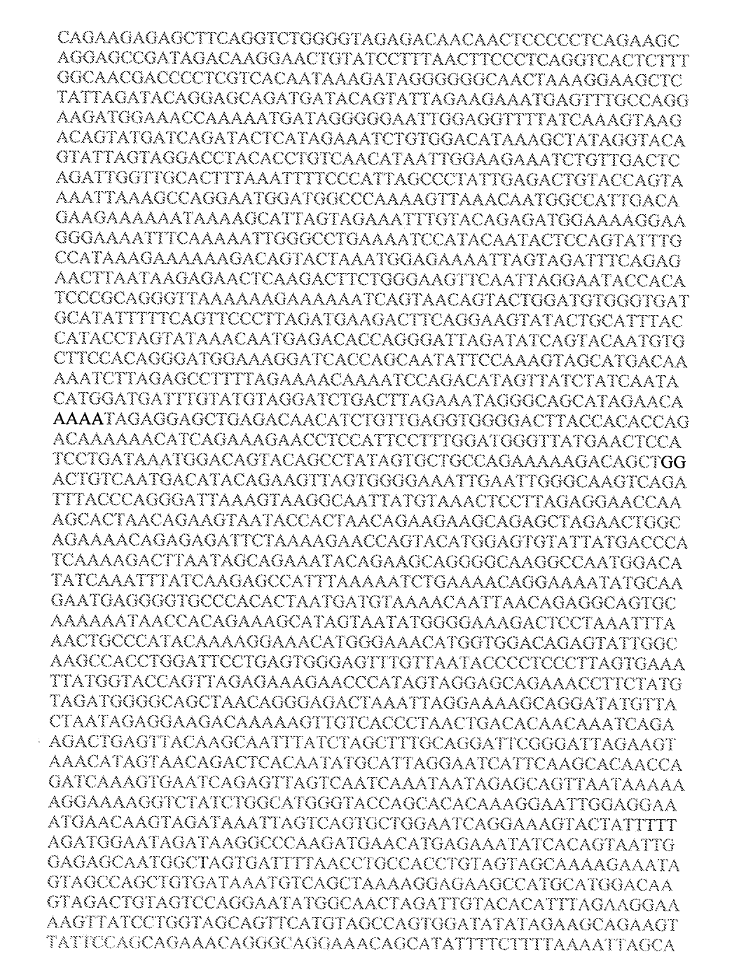

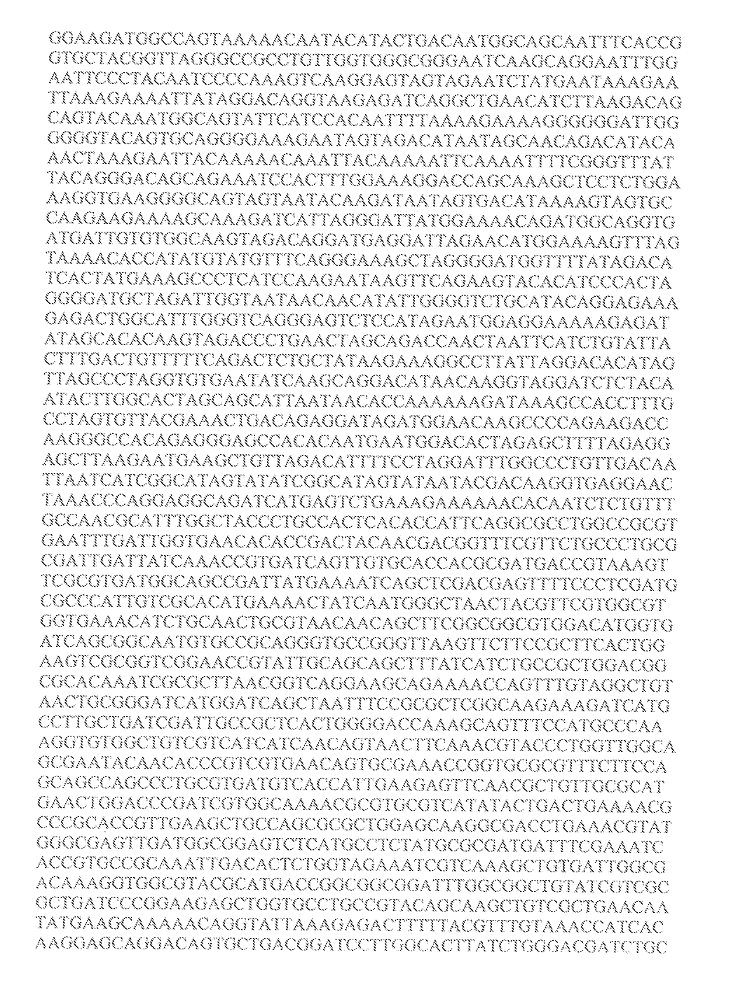

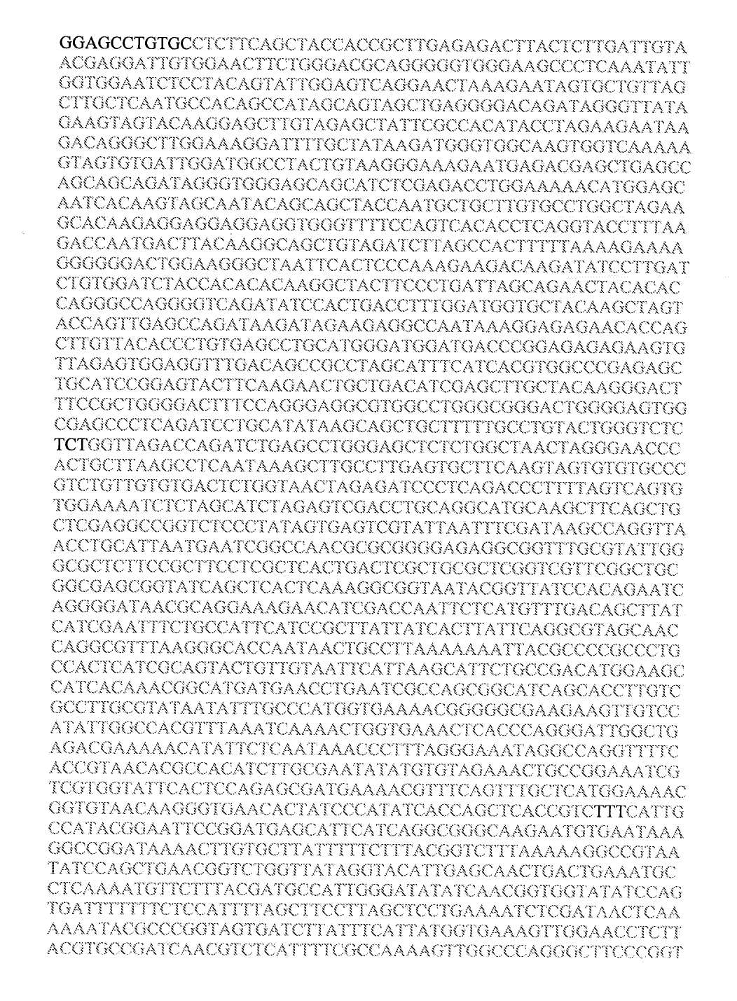

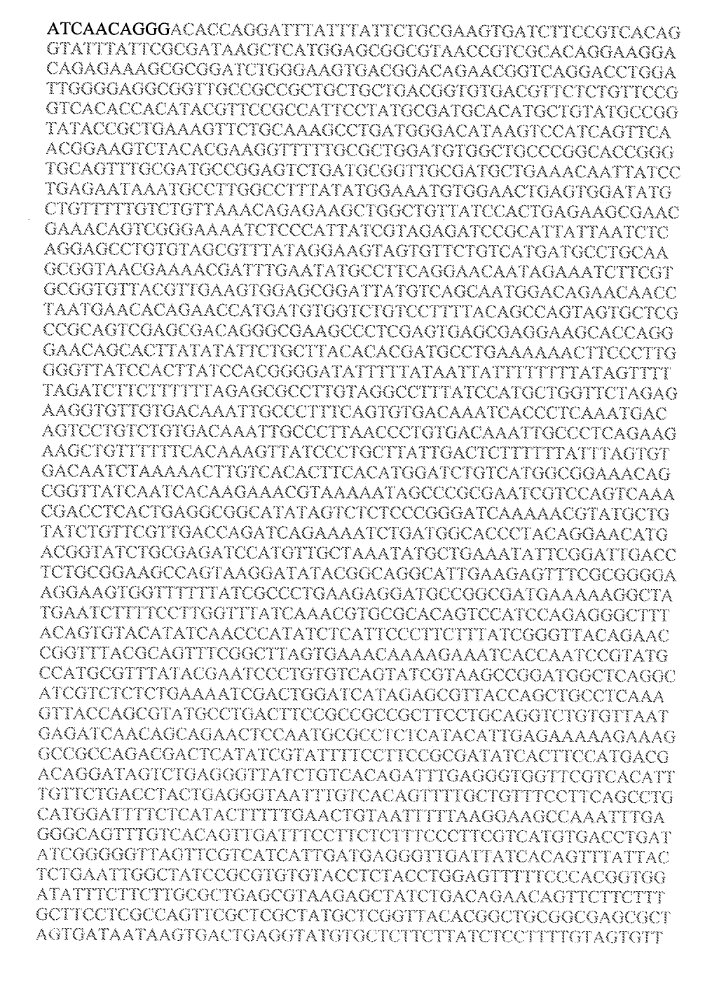

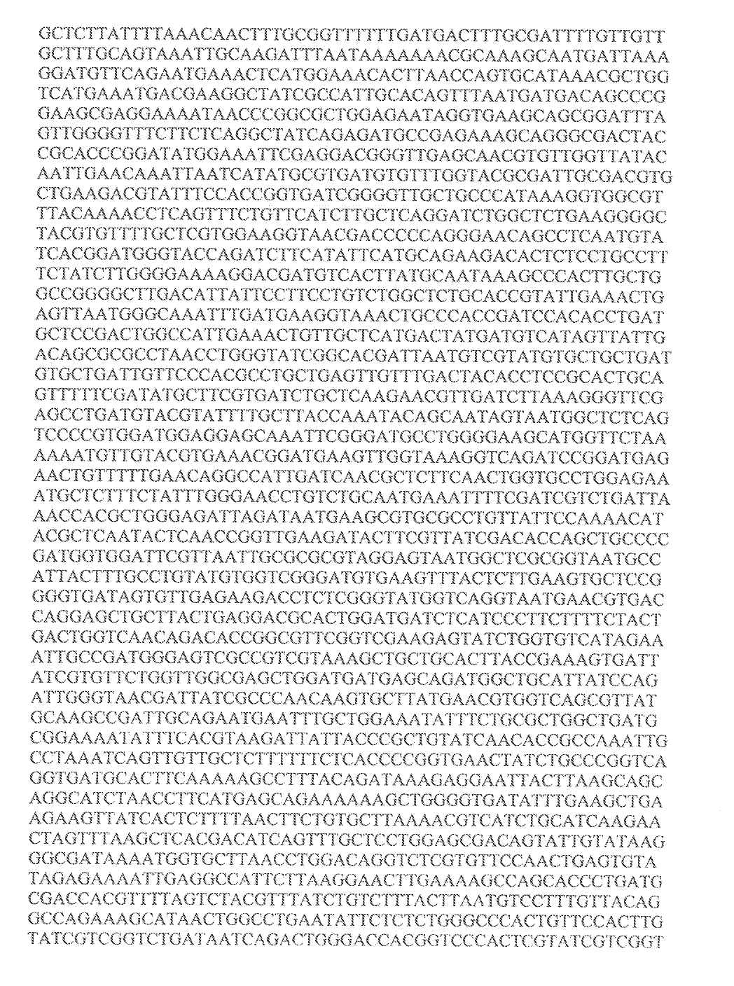

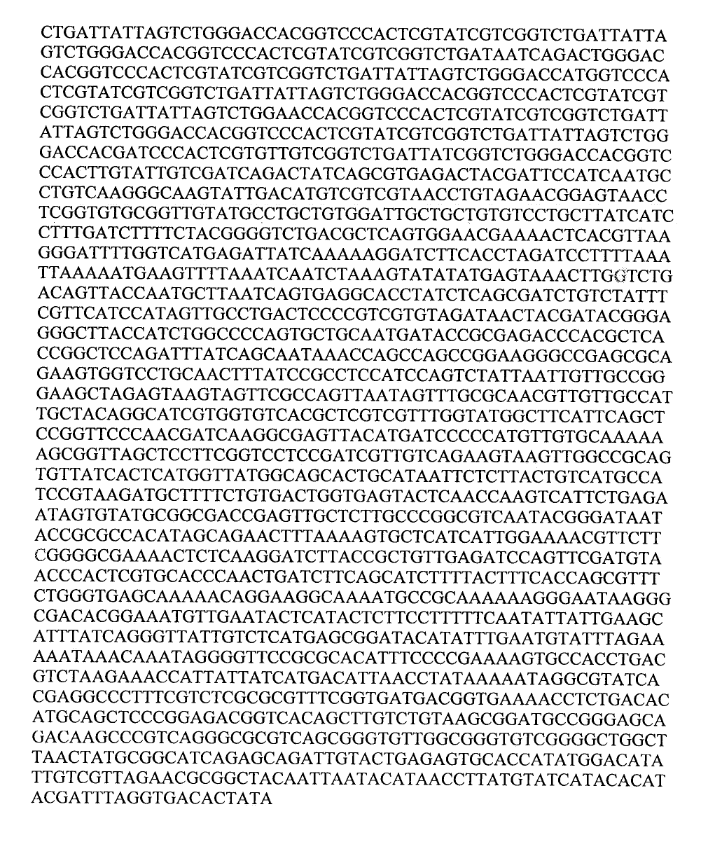

To reconstitute an intact HIV-1 prototype virus by recombineering, an ˜3 kb env sequence was PCR amplified using HIV-1 NL4-3 prototype DNA with flanking targeting homology for our HIV-1 plasmid. This experiment was initially carried out using RIK386 that had been previously transformed with pLL3, thus only the env insert was supplied during the recombineering electroporation. Colonies were chosen and restruck on selective media, so that isolated colonies could be chosen for growth in liquid media, DNA isolation and characterization. Upon visualization in an agarose gel it became apparent that 25% of the clones were running at the correct molecular weight for envelope positive recombinants, while the other 75% were a lower molecular weight plasmid species (the low MW supercoiled plasmid ran at ˜7-7.5 Kb, FIG. 7). The apparent recombination frequency for env⁺ recombinants was 1×10⁻⁵ (only including the 25% that were running at the correct MW).

This experiment was repeated by coelectroporating both pLL3 and the env PCR product in an effort to increase env⁺ recombinants. The logic behind which was, that by introducing the pLL3 plasmid after induction of the Red system, exposure of pLL3 to recombination catalysts during simple BAC replication would be reduced, and initially only intact pLL3 would be available as a recombination target for the env substrate. According to MW ˜20% of the colonies were env⁺ recombinants, while the other 80% were low molecular weight species. The calculated recombination frequency based on colony number is 2×10 ⁻⁴.

Subsequent DNA sequence analysis suggests that the low molecular weight species represent recombinants with primer dimmers formed during PCR. Subsequent recombineering only uses gel-purified PCR product and this is the preferred method. However, this preferred method does not limit the breather of this invention because, as one of skill in the art will appreciate, other methods may be used herein.

Recombineering is a useful tool for manipulation of the HIV genome and construction of an HIV genetic system. The overall recombination frequency of 10⁻⁴-10⁻⁵ for this PCR based recombineering system also allows for the fast generation of thousands of recombinants which will be crucial in future studies when diverse viral sequences are to be sampled from patients.

### Example 4

### Introduction of Positive Negative Selection Marker, galk+ Recombineering

The present invention also uses the method of recombineering which allows genetic engineering in bacteria using homologous recombination. This process is made possible by the expression of bacteriophage λ Red functions. Recombination takes place in a strain of bacteria that expresses 3 proteins from bacteriophage λ Red system: exo, bet and gam. Their functions are respectively, 5′ to 3′ double strand exonuclease, single stand DNA annealing protein, and an inhibitor of E. coil RecBCD enzyme that digests linear DNA. The phage recombination functions are under a bacteriophage λ heat sensitive repressor, cI857, so induction can occur at 42° C. This method is rapid, efficient and provides a viable alternative procedure for cloning any gene in bacteria. The invention uses genetic recombineering to make HIV molecular clones containing the envelope gene of viruses derived from infected children inside the constant backbone of the HXB2 clone.

### EMBODIMENT #1

To generate HIV infectious molecular clones containing envelope genes from HIV infected individuals using recombineering technology we have amplified the envelope genes of viruses corresponding to children infected with HIV using the polymerase chain reaction (PCR). Second, we have introduced the envelope gene together with an env deleted HIV plasmid into bacteria containing the λ Red system. This env deleted plasmid named pLG2, has been recombineered so that in the place of the envelope gene, a galK+ gene has been inserted. Both the PCR products and pLG2 share regions of homology to allow recombination to occur in a precise location. The idea is that through homologous recombination, the galk+ gene will be substituted by the envelope gene derived from viruses from infected children, thus creating a complete infectious molecular clone that differ only in the sequence of the envelope gene. Bacteria containing the envelope gene can grow in minimal glycerol media containing 2-deoxyglucose (DOG), but bacteria containing the galk+ gene would not survive because DOG is toxic for these cells, thus providing a selection for envelope recombinants.

After recombineering, colonies that grew on DOG were tested for the presence of the envelope gene by colony PCR. After analyzing the results we found that in those bacteria where envelope has been effectively inserted instead of galK, there were also plasmids where recombineering has not occurred. This is because the plasmid that is driving the replication of the HIV sequences is pSP73 which has an origin of replication that sustains multiple copies of the plasmids in each cell. In order to use galk as a selection system, we needed to clone HIV into a plasmid with an origin of replication that would maintain a single copy of the plasmid per cell.

This was also achieved by using recombineering. We have exchanged the origin of replication of pSPP73 with the origin of replication of a Bacterial Artificial Chromosome (BAC). This specific BAC, named pBeloBAC11 and available commercially through Invitrogen or NEB, maintains a single copy per cell. We have PCR amplified most of the BAC sequences, with oligonucleotide primers that retain the chloramphenicol resistance gene, the origin of replication, and SopA, SopB and SopC that are important for partitioning and preservation of the plasmid in each daughter cell. These primers have extensions in both 5′ ends that are homologous to sequences on both sides of the pSP73 ori. The recombinant plasmid is resistant to both chloramphenicol and ampicillin, allowing for its positive selection. Subsequent recombineering with the envelope gene should be rapid and efficient.

### EMBODIMENT #2

### To Determine the Degree of Quasispecies Preservation Using Recombineering Technology

To determine quasispecies diversity, PCR products are being cloned and sequenced. Initially, we had some problems cloning the envelope into a vector suitable for sequencing and have discovered that the envelope gene needed to be cloned into a vector that will maintain large DNA molecules. We have obtained ten clones of one patient, 6 clones from another, and cloned the envelope gene from a third.

### Example 5

### Plasmid Maps

The following plasmid maps are provided to further illustrate the invention.

These examples illustrate possible embodiments of the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents. Any headings used herein are provided solely for organizational purposes and are not intended to impart any division or meaning to this document, unless specifically indicated.

All documents cited herein, including websites, journal articles or abstracts,

published or corresponding U.S. or foreign patent applications, issued or foreign patents, or any other documents, are each entirely incorporated by reference herein, including all data, tables, figures, and text presented in the cited document.

The following references are each incorporated herein in their entirety:
1. Coffin, J. M. (1995). HIV population dynamics in vivo: implications for genetic variation, pathogenesis, and therapy. AAAS 267(5197), 483-389.
2. Cohen, A., Clark, A. J., (1986). Synthesis of linear plasmid multimers in Escherichia K-12. J Bacteriology. 167, 327-335.
3. Patel, P., Preston, B., (1994) Marked infidelity of human immunodeficiency virus type 1 reverse transcriptase at RNA and DNA template ends. PNAC 91, 549-553.
4. Quinones-Mateu, M. E., and Arts, E. J. (2006). Virus fitness: concept, quantification, and application to HIV population dynamics. Curr Top Microbiol Immunol 299, 83-140.
5. Quinones-Mateu, M. E., Ball, S. C., Marozsan, A. J., Torre, V. S., Albright, J. L., Vanham, G., van Der Groen, G., Colebunders, R. L., and Arts, E. J. (2000). A dual infection/competition assay shows a correlation between ex vivo human immunodeficiency virus type 1 fitness and disease progression. J Virol 74(19), 9222-33.
6. Stavropoulis, T., Strathdee, G. (2001) Synergy between tetA and rpsL provides high-stringency positive and negative selection in bacterial artificial chromosome Vectors. Genomics 72, 99-104.
7. Thomason, L. C., Costantino, N., Shaw, D. V., and Court, D. L. (2007). Multicopy plasmid modification with phage lambda Red recombineering. Plasmid 58(2), 148-158.
8. Back, N. K., Nijhuis, M. Keufen, W., Boucher, C. A., Oude Essink, B. O., van Kuilenburg, A. B., van Gennip, A. H., and Berkhout, B. (1996). Reduced replication of 3TC-resistant HIV-1 variants in primary cells due to a processivity defect of the reverse transcriptase enzyme. Embo J 15(15), 4040-9.
9. Croteau, G., Doyon, L., Thibeault, D., McKercher, G., Pilote, L., and Lamarre, D. (1997). Impaired fitness of human immunodeficiency virus type 1 variants with high-level resistance to protease inhibitors. J Virol 71(2), 1089-96.
10. Martinez-Picado, J., Savara, A. V., Sutton, L., and D'Aquila, R. T. (1999). Replicative fitness of protease inhibitor-resistant mutants of human immunodeficiency virus type 1. J Virol 73(5), 3744-52.
11. Harrigan, P. R., Bloor, S., and Larder, B. A. (1998), Relative replicative fitness of zidovudine-resistant human immunodeficiency virus type 1 isolates in vitro, J Virol 72(5), 3773-8.
12. Maeda, Y., Venzon, D. J., and Mitsuya, H. (1998). Altered drug sensitivity, fitness, and evolution of human immunodeficiency virus type 1 with pol gene mutations conferring muiti-dideoxynucleoside resistance. J Infect Dis 177(5), 1207-13.
13. Lu, J., Sista, P., Giguel, F., Greenberg, M., and Kuritzkes, D. R. (2004). Relative replicative fitness of human immunodeficieney virus type 1 mutants resistant to enfuvirtide (T-20). J Virol 78(9), 4628-37.
14. Armand-Ugon, M., Quinones-Mateu, M. E., Gutierez, A., Barretina, J., Blanco, J., Schols, D., De Clercq, E., Clotet, B., and Este, J. A. (2003). Reduced fitness of HIV-1 resistant to CXCR4 antagonists. Antivir Ther 8(1), 1-8.
15. Campbell, T. B., Schneider, K., Wrin, T., Petropoulos, C. J., and Connick, E. (2003). Relationship between in vitro human immunodeficiency virus type 1 replication rate and virus load in plasma. J Virol 77(22), 12105-12.
16. Weber, J., Rangel, H. R., Chakraborty, B., Tadele, M., Martinez, M. A., Martinez-Picado, J., Marotta, M. L., Mirza, M., Ruiz, L., Clotet, B., Wrin, T., Petropoulos, C. J., and Quinones-Mateu, M. E. (2003). A novel TaqMan real-time PCR assay to estimate ex vivo human immunodeficiency virus type 1 fitness in the era of multi-target (pol and env) antiretroviral therapy. J Gen Virol 84(Pt 8), 2217-28

1. A method of generating viral recombinants with selected viral gene(s), comprising the steps of
a. introducing said selected viral gene(s) into E. coli containing a plasmid or a defective λ prophage expressing exo, bet and gam, optionally in the presence of a bacteriophage λ heat sensitive cI repressor and a BAC plasmid containing a reference genome for the viral target of recombineering; and
b. exposing said E. coli to conditions such that recombination occurs between the introduced viral gene(s) and the target genome.

2. The method of claim 1, wherein the plasmid comprises a positive or a negative selection marker.

3. The method of claim 1, wherein the plasmid comprises a RTS cassette or a galK⁺ gene.

4. The method of claim 3, wherein the plasmid comprises a RTS cassette.

5. The method of claim 1, wherein the target plasmid has a galK⁺ gene, a RTS cassette, or a stuffer sequence inserted at the site of a deletion that removed the gene that is to be replaced during recombination.

6. The method of claim 1, wherein the target plasmid has a galK⁺ gene, an RTS cassette, or a stuffer sequence inserted at the site of a deletion that removed the gene that is to be replaced during recombination.

7. The method of claim 5 wherein an HIV gene replaces the galK⁺ gene or the stuffer sequence during recombination.

8. The method of claim 1 wherein the plasmid is selected from the group consisting of HIV, Hepatitis A, Hepatitis B, Hepatitis C, polio and influenza viruses.

9. A recombinant virus obtained by the method of claim 1.

10. Use of a recombinant virus of claim 9 to screen for therapeutic agents.

11. A vaccine comprising a recombinant virus of claim 9.

12. Use of a recombinant virus of claim 9 as a tool for measuring vaccine efficacy.

13. Use of a recombinant virus of claim 9 as a tool to measure viral fitness in response to drugs targeting any viral protein.

14. A recombinant library comprising at least one recombinant virus according to claim 9.

15. A recombinant library comprising a plurality of recombinant viruses according to claim 9.

16. Use of the recombinant library according to claim 14 as a tool for measuring vaccine efficacy.

17. Use of the recombinant library according to claim 14 as a tool to measure viral fitness in response to drugs targeting any viral protein.
